(19) United States
(12) Patent Application Publication
(10) Pub. No.: US 2011/0098519 A1
(43) Pub. Date: Apr. 28, 2011

(54) MODIFIED CATALYST COMPOSITION FOR CONVERSION OF ALCOHOL TO ALKENE

(76) Inventors: Kanaparthi Ramesh, Singapore (SG); Armando Borgna, Singapore (SG)

(21) Appl. No.: 12/673,156

(22) PCT Filed: Aug. 12, 2008
(86) PCT No.: PCT/SG08/00296

§ 371 Date: Feb. 11, 2010

Related U.S. Application Data
(60) Provisional application No. 60/935,442, filed on Aug. 13, 2007.

(51) Int. Cl. C07C 1/24; B01J 27/16; B01J 27/02; B01J 23/30; B01J 29/06; B01J 21/04; B01J 21/06; B01J 27/182; B01J 21/08; B01J 29/40; B01J 29/48; B01J 29/076

(52) U.S. Cl. 585/640; 502/208; 502/216; 502/305; 502/60; 502/323; 502/308; 502/309; 502/214; 502/254; 502/311; 502/77; 585/639

(57) ABSTRACT

A catalyst composition for dehydration of an alcohol to prepare an alkene is provided. The catalyst composition comprises a catalyst and a modifying agent which is phosphoric acid, sulfuric acid or tungsten trioxide, or a derivative thereof. A process for preparing an alkene by dehydration of an alcohol is also provided. The process comprises mixing one or more alcohols and optionally water and the catalyst composition.

This application claims the benefit of U.S. Provisional Patent Application Ser. No. 60/935,442, filed Aug. 13, 2007, which is incorporated herein by reference in its entirety.

### FIELD

The invention relates to a catalyst composition comprising a catalyst and a modifying agent. The invention also relates to a catalyst composition comprising a catalyst and a modifying agent which is phosphoric acid, sulfuric acid or tungsten trioxide, or a derivative thereof. The invention further relates to a process for preparing an alkene by dehydration of an alcohol comprising mixing one or more alcohols and optionally water and the catalyst composition. The invention also relates to a process for preparing a catalyst composition comprising adding phosphoric acid to a zeolite.

### BACKGROUND

Light olefins or alkenes, including ethylene, propylene and butenes, are used as chemical intermediates or building blocks in a number of industries, including the petrochemical industry in the production of clean fuels. For example, ethylene is produced in large volumes as a chemical intermediate, and though there are few direct applications for ethylene, it is used as a starting material in the production of various other chemicals, including polyethylene, ethylene oxide, acetaldehyde, ethylbenzene and styrene.

Light olefins or alkenes can be produced by cracking of higher hydrocarbons, for example, steam catalytic cracking of naphtha. It has been reported that the production of light olefins or alkenes by cracking of higher hydrocarbons is energy intensive and accounts for approximately 3.0% of the total energy consumption in the United States of America. For example, it has been reported that ethylene can be commercially produced by thermal catalytic cracking of higher hydrocarbons; however, due to thermodynamic restrictions, this process operates at very high temperatures, including temperatures above 850° C., to achieve reasonable conversions. Under these conditions, however, the ethylene yield is still about 55%. Furthermore, it has been reported that the production of light olefins or alkenes by cracking of higher hydrocarbons accounts for approximately 180 million tons of carbon dioxide (CO₂) emissions world-wide. Decreasing supplies of oil and fossil fuels, their increasing cost mainly due to the high demand for energy, and environmental concerns have driven efforts to develop alternative more environmentally friendly and cost-effective technologies based on renewable sources independent of petroleum.

Several studies have been reported relating to the synthesis of lower olefins (see, for example, R. Le Van Mao and L. H. Dao, U.S. Pat. No. 4,698,452; and C. B. Phillips and R. Datta, Ind. Engl. Chem. Res. 36 (1997) 4466-4475).

Oxidative dehydrogenation of ethane has been reported as an alternative method to produce ethylene at lower temperatures; however, this approach involves the formation of side products along with carbon oxides in the presence of oxygen. Oxidative dehydrogenation of ethane using a Ni—Nb—O mixed oxide catalyst achieving an ethylene yield of 46% has been reported (see, for example, E. Heracleous and A. A. Lemonidou, J. Catal. 237 (2006) 162-174). Selective oxidation of ethanol using mesoporous vanadium-incorporated MCM-41 catalysts to produce ethylene and acetaldehyde has been reported (see, for example, Y. Gucbilmez, T. Dogu and S. Balci, Ind. Eng. Chem. Res. 45 (2006) 3496-3502). It has been reported that the maximum yield of ethylene was about 70% and that the catalyst was deactivated rapidly and reached about 10% conversion in 160 minutes.

Dehydration of an alcohol has been reported as a route to produce olefins and ethers, including the dehydration of ethanol to produce ethylene. Light olefins or alkenes can be produced by the dehydration of lower alcohols, including methanol, ethanol and others (see, for example, P. A. Ruziska, C. D. W. Jenkins, J. R. Lattner, M. P. Nicoletti, M. J. Veraa, and C. F. van Egmond, US Patent Application No. 2006/0149109A1).

It has been reported that ethanol can be produced from renewable sources, including biomass feedstocks such as corn, sugar cane and cellulose. For example, ethanol can be produced as a byproduct of the sugar cane industry. Bioethanol produced via a fermentation process of these sources can be considered as a renewable feedstock, which is independent of fossil fuels, including petroleum. Bioethanol can be used as fuel or as a fuel additive in automotive engines. The production of value-added chemicals from ethanol is also currently attracting considerable interest. In this regard, production of ethylene from ethanol is a promising approach. In particular, production of either aromatic hydrocarbons or light olefins (ethylene, propylene) through the dehydration of ethanol has been the subject of interest. The direct conversion of ethanol to these low olefins is of industrial importance since ethanol can be produced from existing technologies such as fermentation and alternatively from biomass feed stocks.

The following two competing reactions can occur during catalytic dehydration of ethanol to produce ethylene and diethyl ether (DEE):

C₂H₅OH→C₂H₄+H₂O+44.9 kJ/mol (1)

2C₂H₅OH→C₂H₅OC₂H₅+H₂O−25.1 kJ/mol (2)

Dehydration of ethanol to produce ethylene using potassium and silver salts of tungstophosphoric acid as the catalyst has been reported (see, for example, J. Haber, K. Parmin, L. Matachowski, B. Napruszewska, and J. Poltowicz, Journal of Catalysis 207 (2002) 296-306). The highest yield of ethylene was reported at around 70%.

The conversion of ethanol to produce ethylene and diethyl ether using H-ZSM-5 based catalysts has been reported (see, for example, J. Schulz and F. Bandermann, Chem. Eng. Technol. 17 (1994) 179-186). The selective formation of any one of ethylene and DEE has been reported to depend on the reaction temperature, space velocity and Si/Al ratio of the H-ZSM-5 zeolite. Higher Si/Al ratios were reported as favouring the formation of ethylene over DEE. Use of ZSM-5 zeolite modified with Zn or Zn and Mn for the preparation of ethylene from ethanol has been reported (see, for example, R. Le Van Mao and Le H. Dao, U.S. Pat. No. 4,698,452). The maximum yield of ethylene produced was reported at around 88%. Various catalysts, including zeolites (see, for example, W. R. Moser, R. W. Thompson, C.-C. Chiang, and H. Tong, J. Catal. 117 (1989) 19-32), metal oxides, mixed oxides (see, for example, E. A. El-Katatny, S. A. Halawy, M. A. Mohamed and M. I. Zaki, Applied Catalysis A: General 199 (2000) 83-92) and heteropolyacids (see, for example, J. B. McMonagle and J. B. Moffat, J. Catal. 91 (1985) 132-141) have been reported.

It has been reported that methanol can be selectively converted to lower olefins by the addition of phosphorous on H-ZSM-5 catalysts (see, for example, S. A. Butter and W. W. Kaeding, U.S. Pat. No. 3,972,832; and W. W. Kaeding and S. A. Butter, J. Catal. 61 (1980) 155-164).

Generally, the H-ZSM-5 catalyst favours the production of higher hydrocarbons, including benzene, toluene and xylenes. Due to the formation of higher hydrocarbons, the catalyst deactivates quickly and the product distribution shifts towards ethylene. However, it has been reported that after 150 hours, ethylene selectivity decreased to 40% (see, for example, J. Schulz and F. Bandermann, Chem. Eng. Technol. 17 (1994) 179-186). Ethanol conversion over Fe/H-ZSM-5 catalysts to produce C₃ olefins has been reported (see, for example, M. Inaba, K. Murata, M. Saito, I. Takahara, Green Chemistry 9 (2007) 638-646).

Approaches for the selective synthesis of light olefins or alkenes are desired. Catalysts providing improved selectivities towards ethylene during ethanol dehydration are also desired, including catalysts that can be easily and cost-effectively prepared without the use of costly metals as promoters. Versatile catalysts that can be used for the dehydration of other alcohols, including butanol, and of aqueous alcohol solutions are also desired. Catalysts that can perform alcohol dehydration with decreased production of side products are also desired, including catalysts which are durable and exhibit no significant deactivation during reactions.

### SUMMARY

In one broad aspect of the invention, there is provided a catalyst composition comprising a catalyst and a modifying agent.

In still another broad aspect of the invention, there is provided a process for preparing an alkene by dehydration comprising mixing one or more alcohols and optionally water and the catalyst composition of the invention.

In a further broad aspect of the invention, there is provided a process for preparing an alkene by dehydration comprising mixing one or more alcohols and optionally water and a catalyst composition, wherein the catalyst composition comprises a zeolite and a modifying agent, and wherein the modifying agent is an oxide containing compound or an oxy acid.

### BRIEF DESCRIPTION OF THE DRAWINGS

Embodiments of the invention will be discussed with reference to the following Figures:

FIG. 1 graphically illustrates ethanol dehydration on H-ZSM-5 catalyst at 400° C. and 1 atm of pressure.

FIG. 2 graphically illustrates one embodiment of the invention for ethanol dehydration with respect to H₃PO₄ content on H-ZSM-5 catalyst at 400° C. and 1 atm of pressure.

FIG. 3 graphically illustrates another embodiment of the invention for ethanol dehydration on 20 wt % H₃PO₄ on H-ZSM-5 catalyst at 400° C. and 1 atm of pressure.

FIG. 4 graphically illustrates a further embodiment of the invention for ethanol dehydration on 20 wt % H₃PO₄ on H-ZSM-5 catalyst with respect to various space velocities at 300° C. and 1 atm of pressure.

FIG. 5 graphically illustrates a further embodiment of the invention for butanol dehydration on 20 wt % H₃PO₄ on H-ZSM-5 catalyst at 325° C. and 1 atm of pressure.

FIG. 6 graphically illustrates another embodiment of the invention for butanol dehydration on 20 wt % H₃PO₄ on H-ZSM-5 catalyst at 325° C. and 1 atm of pressure.

### DETAILED DESCRIPTION

The invention relates to a catalyst composition for the conversion of an alcohol to an alkene.

In an embodiment of the invention, the catalyst composition may be, for example, and without limitation, a solid catalyst. In an embodiment, the catalyst composition may be, for example, and without limitation, a solid acid catalyst. In an embodiment, the catalyst composition may comprise, for example, and without limitation, a catalyst and a modifying agent. The expression “modifying” or “modified” and the like would be understood by those of ordinary skill in the art to include all forms of physical and chemical interactions between the catalyst and the modifying agent, and may include, for example, and without limitation, “impregnated”, “incorporated”, “supported”, “loaded”, “added”, “placed”, “anchored” and the like. The expression “impregnated” would be understood by those of ordinary skill in the art, and may include the situation where the modifying agent is anchored on a support using the pores and/or surface of the support. In an embodiment, the modifying agent may be, for example, anchored to a surface of the catalyst and/or impregnated within pores of the catalyst.

In an embodiment of the invention, the modifying agent may be, for example, and without limitation, an oxide containing compound or an oxy acid. These compounds are believed to enhance the surface acidity. However, some oxides may also enhance the oligomerization of light olefins to produce bulky molecules and thus the selectivity for alkene production may drop considerably. Also, some metal oxides may be accompanied with surface oxygen atoms which help in oxidizing the reactant or products resulting in non-selective product formation. Moreover, metal or metal oxides may leach from the surface after a period of time. These factors should be considered when selecting an oxide containing compound or oxy acid. Some examples of the modifying agent of the present invention include phosphate or sulfate compounds such as phosphoric acid or sulfuric acid, or a derivative thereof, or a transition metal oxide or a derivative thereof, including tungsten trioxide, ZrO₂ and MoO₃. In one embodiment, for example, H₃PO₄, H₂PO₄⁻, HPO₄²⁻, PO₄³⁻, H₂SO₄, HSO₄⁻, SO₄²⁻ or WO₃ could be used. In an embodiment, the modifying agent may be, for example, phosphoric acid or a derivative thereof. In another embodiment, ammonium dihydrogen phosphate could be used to modify the catalyst.

In an embodiment of the invention, the catalyst modified with the modifying agent may be, for example, and without limitation, a bulk oxide or zeolite catalyst.

In an embodiment, the catalyst modified with the modifying agent may be, for example, and without limitation, a bulk oxide. Suitable bulk oxides would be understood to and can be determined by those of ordinary skill in the art. In an embodiment, the catalyst may be, for example, and without limitation, a pure bulk oxide. In an embodiment, the bulk oxide may be, for example, and without limitation, alumina, zirconia, titania, silica or niobia.

In an embodiment of the invention, the catalyst modified with the modifying agent may be, for example, and without limitation, a zeolite. The meaning of the expression “zeolite” would be understood to those of ordinary skill in the art. A zeolite may include, for example, and without limitation, a hydrated aluminosilicate of the alkaline and alkaline earth metals. Suitable zeolites would be understood to and can be determined by those of ordinary skill in the art. In an embodiment of the invention, the zeolite may be, for example, and without limitation, of natural or synthetic origin. In an embodiment, the zeolite may be, for example, and without limitation, crystalline. In an embodiment, the zeolite may be, for example, and without limitation, a pentasil-type zeolite. In an embodiment, the zeolite may be, for example, and without limitation, HY, H-BETA, H-Mordenite or ZSM-5 zeolite. The expressions “HY”, “H-BETA”, “H-Mordenite” and “ZSM-5” would be understood to those of ordinary skill in the art. In an embodiment, the zeolite may be, for example, and without limitation, ZSM-5 zeolite. The expression “ZSM-5” is used interchangeably with the expression “H-ZSM-5” throughout this entire specification.

In an embodiment, the catalyst composition may comprise, for example, and without limitation, H-ZSM-5 zeolite and a modifying agent which is phosphoric acid or a derivative thereof. The H-ZSM-5 zeolite is described in R. J. Argauer and G. R. Landolt, U.S. Pat. No. 3,702,886, which is incorporated herein by reference in its entirety. The H-ZSM-5 zeolite, which may be used to prepare the phosphoric acid modified H-ZSM-5 catalyst of the invention can be made and characterized in accordance with this reference.

In an embodiment of the invention, the zeolite may have, for example, and without limitation, a silica/alumina (Si/Al) ratio of from less than about 280, from less than about 40, from greater than about 20 to about 280, from greater than about 20 to about 40, and including any specific value within these ranges, such as, for example, about 25 or about 30.

In an embodiment of the invention, the catalyst composition may have, for example, and without limitation, an average particle size of less than about 500 μm, less than about 450 μm, less than about 425 μm, less than about 400 μm, less than about 350 μm, less than about 300 μm, less than about 250 μm, from about 70 to about 400 μm, from about 200 to about 500 μm, from about 200 to about 400 μm, from about 250 to about 425 μm, and including any specific value within these ranges, such as, for example, and without limitation, about 250 μm.

In an embodiment of the invention, the catalyst composition may be, for example, and without limitation, unactivated. In an embodiment, the catalyst composition may be, for example, and without limitation, activated. Means for activating the catalyst composition would be understood to and can be determined by those of ordinary skill in the art. In an embodiment, the catalyst composition may be activated, for example, and without limitation, by treating the catalyst composition at an elevated temperature. In an embodiment, for example, and without limitation, the catalyst composition may be treated in nitrogen (N₂) at about 400 to about 500° C., and including any specific temperature within this range, for about 1 to about 5 hours, and including any specific value within this range, such as, for example, about 2 or about 5 hours.

The amount of modifying agent anchored and/or impregnated in the catalyst is not particularly limited and suitable amounts of the modifying agent would be understood to and can be determined by those of ordinary skill in the art. In an embodiment of the invention, the amount of modifying agent anchored and/or impregnated in the catalyst may include, for example, and without limitation, from greater than about 0.1 wt %, from greater than about 1.0 wt %, from greater than about 5 wt %, from greater than about 10 wt %, from greater than about 15 wt %, from greater than about 20 wt %, from greater than about 30 wt %, from greater than about 40 wt % and from greater than about 50 wt %. In an embodiment, the amount of modifying agent anchored and/or impregnated in the catalyst may include, for example, and without limitation, from less than about 50 wt %, from less than about 25 wt %, from less than about 20 wt %, from less than about 15 wt %, from less than about 10 wt %, from less than about 5 wt %, from about 1 to about 50 wt %, from about 1 to about 20 wt %, from about 5 to about 50 wt %, from about 5 to about 20 wt %, and including any specific value within these ranges, such as, for example, about 5 wt %, about 7 wt % or about 20 wt %. The amounts of modifying agent refer to the wt % of modifying agent in the catalyst.

In an embodiment of the invention, the process for preparing the catalyst composition may comprise, for example, and without limitation, adding a modifying agent to a catalyst. In an embodiment, the process for preparing the catalyst composition may comprise, for example, and without limitation, adding phosphoric acid to a zeolite catalyst. Without being bound by theory, it is believed that phosphoric acid may be used as a promoter to tailor the acidic properties of H-ZSM-5. In an embodiment, the process for preparing the catalyst composition may comprise, for example, and without limitation, impregnating the zeolite catalyst with phosphoric acid. In an embodiment, the process for preparing the catalyst composition may comprise, for example, and without limitation, adding phosphoric acid to the zeolite by an impregnation method. In an embodiment, the impregnation method may be, for example, and without limitation, a dry impregnation method. The meaning of the expression “dry impregnation” would be understood to those of ordinary skill in the art. Dry impregnation may include, for example, and without limitation, impregnation which uses an amount of water which is less than or equal to that required to fill the pores of the substrate. In an embodiment, for example, and without limitation, impregnation may include using an amount of water which is greater than that required to fill the pores of the substrate. In an embodiment, phosphoric acid may be added, for example, and without limitation, in an amount of about 5.0 to about 20.0 wt %. The amount of phosphoric acid refers to the wt % of phosphoric acid in the zeolite. In an embodiment, the process for preparing the catalyst composition may comprise, for example, and without limitation, treating the zeolite at an elevated temperature before adding the phosphoric acid. In an embodiment, the process for preparing the catalyst composition may comprise, for example, and without limitation, treating the zeolite at about 500° C. for about 6 hours in static air before adding the phosphoric acid. In an embodiment, the process for preparing the catalyst composition may comprise, for example, and without limitation, grinding and/or pelletizing the catalyst composition.

In an embodiment of the invention, the catalyst composition has, for example, and without limitation, a pore volume of less than about 0.25 cc/g, of less than about 0.22 cc/g, of less than about 0.20 cc/g, of less than about 0.15 cc/g, of less than about 0.12 cc/g, from about 0.10 cc/g to about 0.25 cc/g, from about 0.10 cc/g to about 0.22 cc/g, from about 0.10 cc/g to about 0.17 cc/g, and including any specific value within these ranges.

In an embodiment of the invention, the catalyst composition has, for example, and without limitation, a surface area of from less than about 350 m²/g, less than about 300 m²/g, less than about 250 m²/g, less than about 200 m²/g, less than about 150 m²/g, less than about 100 m²/g, less than about 75 m²/g, less than about 70 m²/g, less than about 50 m²/g, from about 70 to about 350 m²/g, from about 70 to about 300 m²/g, from about 70 to about 250 m²/g, from about 70 to about 200 m²/g, and including any specific value within these ranges.

The catalyst compositions as described anywhere above may be used to catalyze, for example, and without limitation, conversion or dehydration of an alcohol to an alkene.

In an embodiment of the invention, the process for preparing an alkene may comprise, for example, and without limitation, mixing one or more alcohols and optionally water and the catalyst composition as defined anywhere above.

In an embodiment of the invention, the alcohol may be, for example, and without limitation, an alkyl alcohol. For example, and without limitation, the alcohol may be a C₁₋₆ alkyl alcohol. The C₁₋₆ alkyl group of the C₁₋₆ alkyl alcohol may be, for example, and without limitation, any straight or branched alkyl, for example, methyl, ethyl, n-propyl, i-propyl, sec-propyl, n-butyl, i-butyl, sec-butyl, t-butyl, n-pentyl, i-pentyl, sec-pentyl, t-pentyl, n-hexyl, i-hexyl, 1,2-dimethylpropyl, 2-ethylpropyl, 1-methyl-2-ethylpropyl, 1-ethyl-2-methylpropyl, 1,1,2-trimethylpropyl, 1,1,2-triethylpropyl, 1,1-dimethylbutyl, 2,2-dimethylbutyl, 2-ethylbutyl, 1,3-dimethylbutyl, 2-methylpentyl or 3-methylpentyl. In an embodiment, the alcohol may be, for example, and without limitation, methanol, ethanol, n-butanol or a combination thereof. In an embodiment, the alcohol may be, for example, and without limitation, ethanol, n-butanol, a combination of methanol and ethanol or a combination of ethanol and n-butanol. In an embodiment, the alcohol may be, for example, ethanol. In an embodiment, the alcohol may be, for example, n-butanol. In an embodiment, the alcohol may be, for example, a combination of methanol and ethanol. In an embodiment, the alcohol may be, for example, a 1:1 mixture of methanol and ethanol. In an embodiment, the alcohol may be, for example, a combination of ethanol and n-butanol. In an embodiment, the alcohol may be, for example, a 1:1 mixture of ethanol and n-butanol.

Suitable sources for the alcohol of the invention would be understood to or can be determined by those of ordinary skill in the art. In an embodiment, the alcohol may be, for example, and without limitation, obtained from a synthesis gas, biomass or a biofuel. In an embodiment, the alcohol may be, for example, and without limitation, obtained from biomass or a biofuel.

In an embodiment of the invention, for example, and without limitation, one or more alkenes may be prepared. In an embodiment, the alkene may be, for example, and without limitation, a C₂₋₆ alkene. In an embodiment, the C₂₋₆ alkene may be, for example, and without limitation, any straight or branched alkene, for example, ethylene, propylene, but-1-ene, cis-but-2-ene, trans-but-2-ene, isobutylene, pent-1-ene, cis-pent-2-ene, trans-pent-2-ene, 2-methylbut-1-ene, isopentene or 2-methylbut-2-ene. In an embodiment, the alkene may be, for example, and without limitation, ethene, 1-butene, isobutylene, trans-2-butene, propylene or a combination thereof. In an embodiment, the alkene may be, for example, ethene.

The amount of the alcohol which may be used in the process is not particularly limited. Suitable amounts of the alcohol would be understood to and can be determined by those of ordinary skill in the art.

In an embodiment of the invention, the process may comprise, for example, and without limitation, mixing one or more alcohols and water and the catalyst composition as defined anywhere above. The concentration of alcohol in the solution is not particularly limited, and suitable concentrations would be understood to and can be determined by those of ordinary skill in the art. In an embodiment, the concentration of alcohol in the solution may be, for example, and without limitation, from about 10 to about 100 vol %, from about 25 to about 100 vol %, and including any specific value within these ranges.

Suitable amounts of the catalyst composition which may be used would be understood to and can be determined by those of ordinary skill in the art.

The reaction temperature is not particularly limited and suitable reaction temperatures would be understood to and can be determined by those of ordinary skill in the art. In an embodiment, the reaction temperature may be, for example, and without limitation, from about 200 to about 500° C., from about 250 to about 500° C., from about 350 to about 500° C., from about 250 to about 450° C., from about 350 to about 450° C., and including any specific value within these ranges, such as, for example, about 250° C., about 300° C., about 325° C., about 350° C., about 400° C. or about 450° C.

The reaction time is not particularly limited and suitable reaction times would be understood to and can be determined by those of ordinary skill in the art.

In an embodiment, the dehydration reaction may be, for example, and without limitation, conducted in the gas or vapour phase. The dehydration reaction could also be conducted in the liquid phase, depending on the alcohol and other reaction conditions.

The pressure of the dehydration reaction is not particularly limited, and suitable pressures would be understood to and can be determined by those of ordinary skill in the art. In an embodiment, the dehydration reaction may be, for example, and without limitation, conducted at a total pressure of about 1 atm or about atmospheric pressure.

In an embodiment, the dehydration reaction may be conducted, for example, and without limitation, in a flow reactor. In an embodiment, the dehydration reaction may be conducted, for example, and without limitation, in a fluid or fluidized bed reactor. The flow reactor is not particularly limited, and suitable flow reactors would be understood to and can be determined by those of ordinary skill in the art. In an embodiment, the flow reactor may be, for example, and without limitation, a fixed bed reactor, a continuous flow fixed bed reactor, or a down flow fixed bed reactor.

The weight hourly space velocity (WHSV, h⁻¹) of the dehydration reaction is not particularly limited and suitable weight hourly space velocities would be understood to and can be determined by those of ordinary skill in the art. The expression “weight hourly space velocity” would be understood to those of ordinary skill in the art, and may include, for example, and without limitation, the mass of reactants (g) treated per amount of catalyst (g) per hour. In an embodiment, the dehydration reaction may be conducted with, for example, and without limitation, a weight hourly space velocity of less than about 19 h⁻¹, from about 4 to about 19 h⁻¹, and including any specific value within these ranges.

In an embodiment of the invention, for example, and without limitation, a diluent may be used for the alcohol or alcohol containing medium. The diluent is not particularly limited and suitable diluents would be understood to and can be determined by those of ordinary skill in the art. In an embodiment, the diluent may be, for example, and without limitation, non-reactive to the reactants and the catalyst composition. In an embodiment, the diluent may be, for example, and without limitation, helium.

It has been observed that a catalyst composition of the invention exhibited a high degree of stability during a dehydration reaction, including a catalyst composition where deactivation was not detected for longer than about 20 hours, longer than about 21 hours, longer than about 25 hours, longer than about 50 hours, longer than about 60 hours, longer than about 110 hours, longer than about 150 hours, longer than about 250 hours, and including for any specific value within these ranges, such as, for example, longer than about 110, about 150 or about 250 hours. It was observed that a catalyst composition of the invention did not require regeneration during a dehydration reaction.

In an embodiment of the invention, the catalyst composition may be, for example, and without limitation, regenerated. Suitable methods for regenerating the catalyst composition would be understood to and can be determined by those of ordinary skill in the art.

It has been observed that a catalyst composition of the invention exhibited a high degree of alcohol conversion, including an alcohol conversion of greater than about 25 mol %, greater than about 45 mol %, greater than about 75 mol %, greater than about 85 mol %, greater than about 90 mol %, greater than about 95 mol %, greater than about 96 mol %, greater than about 97 mol %, greater than about 98 mol %, greater than about 99 mol %, from about 50 to about 99.9 mol %, from about 90 to about 99.9 mol %, from about 95 to about 99.9 mol %, from about 96 to about 99.9 mol %, from about 97 to about 99.9 mol %, from about 98 to about 99.9 mol %, from about 99.0 to about 100 mol %, and including any specific value within these ranges, such as, for example, about 90 mol %, about 95 mol % or about 99 mol %.

It has been observed that a catalyst composition of the invention exhibited a high alkene yield, including an alkene yield of greater than about 80 mol %, greater than about 85 mol %, greater than about 90 mol %, greater than about 95 mol %, greater than about 96 mol %, greater than about 97 mol %, greater than about 98 mol %, greater than about 99 mol %, from about 90 to about 99.9 mol %, from about 95 to about -99.9 mol %, from about 96 to about 99.9 mol %, from about 97 to about 99 mol %, from about 98 to about 99 mol %, and including any specific value within these ranges, such as, for example, about 85 mol % or about 98 mol %.

The dehydration reaction may further produce, for example, and without limitation, other products including an ether and aromatics. In an embodiment, the ether produced may be, for example, and without limitation, diethyl ether (DEE). It has been observed that a catalyst composition of the invention exhibited selectivity towards an alkene, including an alkene selectivity of from greater than about 35 mol %, greater than about 65 mol %, greater than about 75 mol %, greater than about 85 mol %, greater than about 95 mol %, greater than about 97 mol %, greater than about 98 mol %, greater than about 99 mol %, from about 99.0 to about 99.9 mol % and including any specific value within these ranges, such as, for example, about 97 mol %, about 98 mol % or about 99 mol %. It has been observed that a catalyst composition of the invention exhibited a high degree of selectivity towards ethylene, including an ethylene selectivity of greater than about 97 mol %, greater than about 98 mol %, and including any specific value within these ranges.

Those of ordinary skill in the art will appreciate that the process may comprise, for example, and without limitation, optionally separating, isolating or purifying the product from the reaction mixture. Suitable separation, isolation and purification methods would be understood to and can be determined by those of ordinary skill in the art.

### EXAMPLES

### A) Preparation of Catalyst

H-ZSM-5 was obtained by treating commercial NH₄-ZSM-5 (Zeolyst™, CBV, Si/Al 30) at 500° C. for 6 hours in air.

5.0 to 20.0 wt % of H₃PO₄ was added to the H-ZSM-5 by an impregnation method.

The catalysts were mixed thoroughly and pelletized to obtain an average particle size of 250-425 μm.

The surface properties of H₃PO₄ modified and unmodified catalysts are presented in Table 1.

TABLE 1
Surface properties of unmodified ZSM-5
and H₃PO₄ modified ZSM-5 catalysts

| | Surface area | Pore volume | Pore width |
|---|---|---|---|
| Catalyst | (m²/g) | (cc/g) | (Å) |
| H-ZSM-5 | 366 | 0.26 | 14.5 |
| 5HP-ZSM-5 | 276 | 0.21 | 14.6 |
| 10HP-ZSM-5 | 199 | 0.16 | 14.5 |
| 20HP-ZSM-5 | 74 | 0.12 | 15.5 |

Note:
Surface area and pore volume derived from BET and BJH methods; pore width derived from Horvath-Kawazoe method.

H-ZSM-5 and 20HP-ZSM-5 catalysts were studied by NH₃ temperature programmed desorption (NH₃-TPD) in the temperature range of 55-900° C. Two peak maximums were observed for the H-ZSM-5 sample at 227 and 476° C. Without being bound by theory, it is believed that this suggests that two types of acidic sites are present. The deconvolution of these peaks yielded areas of 72% and 28%, respectively. One broad peak with a peak maximum around 203° C. was observed for 20HP-ZSM-5 catalyst. The deconvolution of this broad peak centred at 203 and 476° C. resulting in peaks having areas of 88% and 12%, respectively. The total peak area of 20HP ZSM-5 was reduced by 8% compared to H-ZSM-5. Without being bound by theory, it is believed that this suggests that there is a decrease in the total acidity of ZSM-5 with H₃PO₄ modification.

### B) Preparation of Alkene

### Comparative Example 1

Dehydration of ethanol over unmodified H-ZSM-5 catalyst was conducted. The reaction was carried out at 400° C. and 1 atm of pressure.

About 0.5 g of the catalyst (average particle size 250 μm) was diluted with an equal amount of quartz grains and charged in a down flow fixed bed steel reactor.

The reactor used for the process of ethanol conversion has three zones: firstly, a zone where 3 mm size glass beads pre-heat and mix the ethanol and helium homogeneously; secondly, a catalyst zone where the reactant feed, in a vaporized form, contacts the catalyst; and thirdly, a post reaction zone. The reaction temperatures of the three zones were maintained by pre-calibrated thermal heaters and the catalyst temperature was monitored by a thermocouple inside the reactor.

Prior to the reaction, the catalysts were activated in situ in N₂ gas at 500° C. for about 5 hours.

Catalytic tests were performed by injecting the alcohol (including ethanol or diluted ethanol solution) with an Agilent™ HPLC infusion pump with a fixed flow rate varying from 0.025 ml/min to 0.5 ml/min, via a preheater operating at 175° C. Helium was used as a diluent gas and connected in-line with a flow meter. Product and reactant distributions were monitored by online GC (HP 6890 Series) using a HP-PONA™ column. The lines were heated to avoid any condensation. The representative samples condensed at low temperatures were also analyzed by using GC-MSD for product identification.

Conversion of ethanol and selectivity towards ethylene were calculated according to the following equations.

$$\begin{matrix}{X}_{EtOH} = \frac{{N}_{EtOH} , i - {N}_{EtOH} , j}{{N}_{EtOH} , i} \times 100 & ( 3 ) \\ {S}_{E} = \frac{{N}_{ɛ} , j}{\sum N , i} \times 100 & ( 4 )\end{matrix}$$

X_{EtOH}=% molar conversion of ethanol, N_{EtOH}, i is the number of moles of ethanol introduced, N_{EtoH}, j is the number of moles of ethanol observed in the products, S_{E} is the selectivity towards ethylene in % moles, N_{E}, j is the number of moles of ethylene observed in the products, and Σ_{N}, i is the total number of moles of products observed during the reaction. Those of ordinary skill in the art will appreciate that these equations are not limited to calculating conversion of ethanol and selectivity towards ethylene, and that conversions of other alcohols and selectivities towards other products can also be calculated according to these equations.

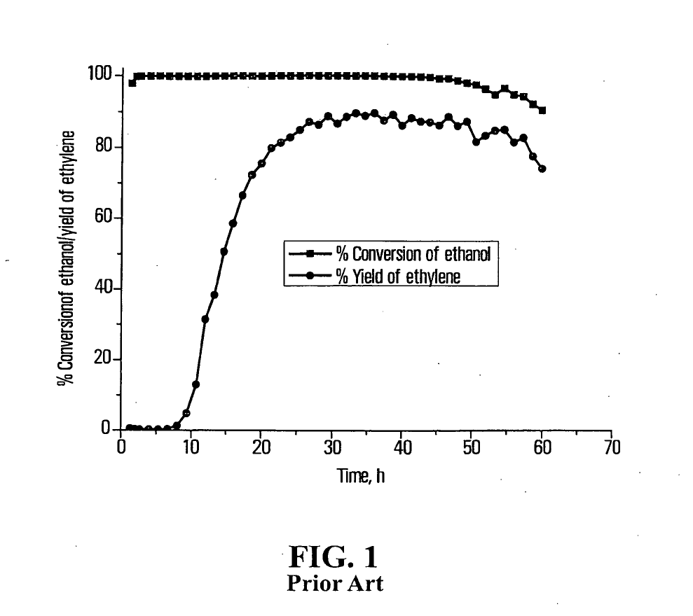

FIG. 1 shows the activity results for the reaction at 400° C. over the H-ZSM-5 catalyst at 1 atm of pressure. There was no ethylene observed before 10 hours. About 150 compounds were detected by the GC before 10 hours. Ethylene yield was observed to start to increase after this time and reach a maximum of 90%, and to steadily decrease after 50 hours. After 60 hours, ethanol yield was observed to reach 78%. A corresponding increase in diethyl ether selectivity was observed. A high ethanol conversion of up to 99.9% was observed for the catalyst. The ethanol conversion was observed to remain the same for 25 hours. Deactivation thereafter of the catalyst over time was observed. The decrease in ethanol conversion was observed to be more prominent after 50 hours.

### Comparative Example 2

Dehydration of ethanol was conducted over unmodified H-ZSM-5 catalyst under similar reaction conditions as those described in Comparative Example 1 with varying reaction temperatures. The results of ethanol dehydration on H-ZSM-5 catalyst with respect to reaction temperature are presented in Table 2. An increase in the activity of the unmodified H-ZSM-5 catalyst with reaction temperature was observed, and highest conversion was observed at 400° C. A decrease in conversion with a further increase in reaction temperature was observed.

TABLE 2
Results of ethanol dehydration activity with
reaction temperature over H-ZSM-5 catalyst^{a}

| Reaction | | | % Selectivity |
|---|---|---|---|
| temperature | % Conversion | % Selectivity | of diethyl |
| (° C.) | of ethanol | of ethylene | ether |
| 300 | 92 | 80 | 12 |
| 400 | 94 | 89 | 4 |
| 500 | 88 | 74 | 3 |

^{a}Reaction Conditions: 0.5 g catalyst, 1 atm pressure, WHSV 18.36 h⁻¹.

### Example 1

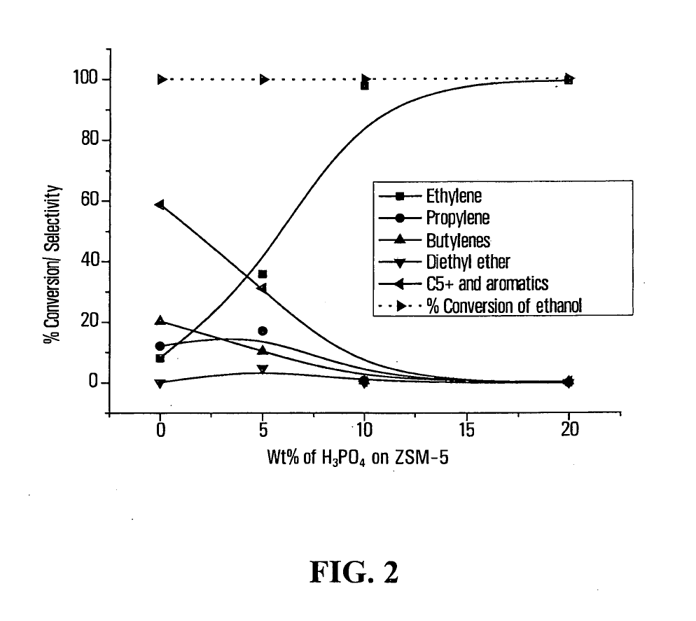

Dehydration of ethanol was conducted over H₃PO₄ modified H-ZSM-5 catalysts with varying amounts of H₃PO₄ under similar reaction conditions to those described in Comparative Example 1. H₃PO₄ modified H-ZSM-5 catalysts were prepared according to the protocol described above under heading A. FIG. 2 shows the activity results of the reaction at 400° C. and 1 atm of pressure. Results for dehydration of ethanol over unmodified H-ZSM-5 catalyst are included in FIG. 2 for comparison purposes. A trend was observed, where at low H₃PO₄ contents, side products including propylene, aromatics and C₅₊ hydrocarbons were observed. With increasing H₃PO₄ content, the selectivity towards ethylene was observed to increase and reach almost 98% for 20 wt % H₃PO₄ modified ZSM-5 catalyst.

### Example 2

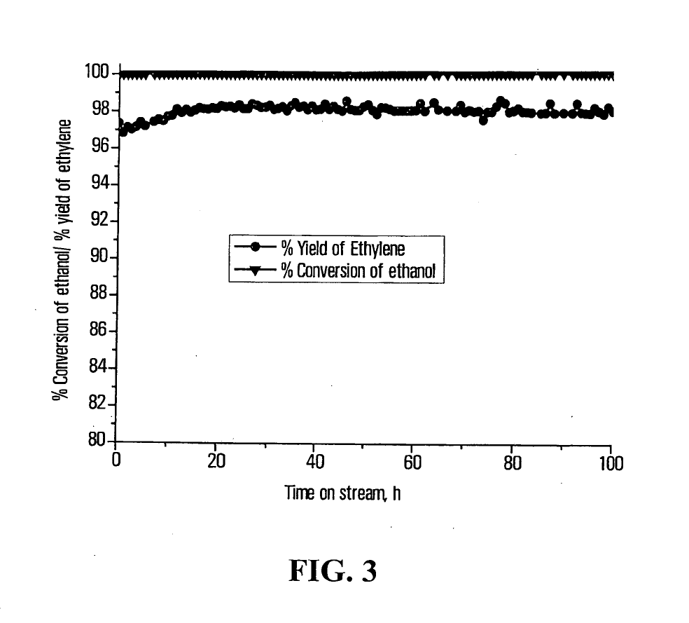

Dehydration of ethanol over H₃PO₄ (20 wt %) modified H-ZSM-5 catalyst was conducted under similar reaction conditions to those described in Comparative Example 1 with increasing reaction temperature from 250 to 450° C. H₃PO₄ modified H-ZSM-5 catalysts were prepared according to the protocol described above under heading A. The results of ethanol dehydration activity on the H₃PO₄/H-ZSM-5 catalyst are presented in Table 3. An increase of the conversion of ethanol from 50% to 99% was observed with the increase in reaction temperature. An increase of the ethylene selectivity with an increase in reaction temperature was also observed. Ethanol dehydration activity on H₃PO₄ (20wt %) on H-ZSM-5 catalyst at 400° C. and 1 atm of pressure is shown in FIG. 3. As can be seen from FIG. 3, no deactivation was observed after running for 110 hours.

TABLE 3
Results of ethanol dehydration activity with reaction temperature
over (20 wt %) H₃PO₄ impregnated H-ZSM-5 catalyst^{a}

| Reaction | | | % Selectivity |
|---|---|---|---|
| temperature | % Conversion | % Selectivity | of diethyl |
| (° C.) | of ethanol | of ethylene | ether |
| 250 | 25.35 | 3.46 | 96.54 |
| 300 | 75.44 | 35.46 | 64.2 |
| 350 | 85.66 | 88.53 | 11.26 |
| 400 | 99.73 | 99.0 | 0.015 |
| 450 | 99.96 | 98.71 | 0.031 |

^{a}Reaction Conditions: 0.5 g catalyst, 1 atm pressure, WHSV 18.36 h⁻¹.

### Example 3

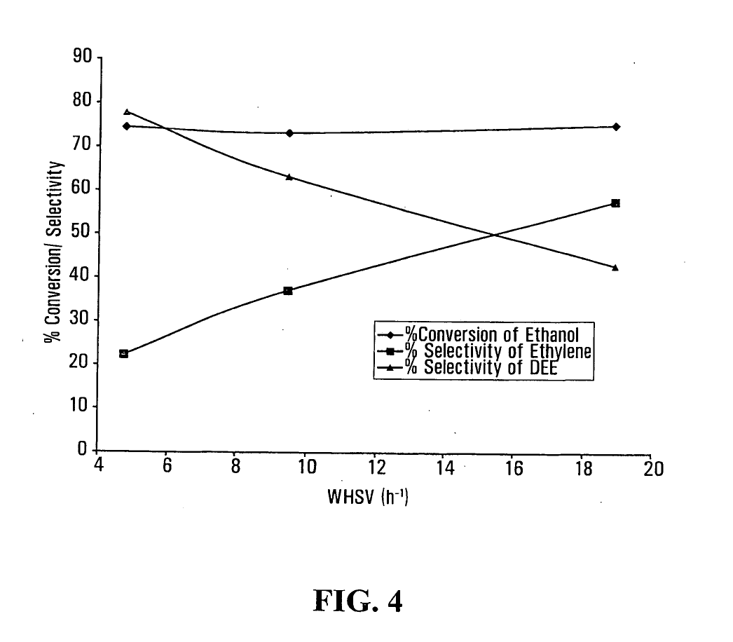

Dehydration of ethanol over H₃PO₄ (20 wt %) modified H-ZSM-5 catalyst was conducted under similar reaction conditions to those described in Comparative Example 1 with varying WHSV of ethanol from 4.73 to 18.9 h⁻¹ at300° C. H₃PO₄ modified H-ZSM-5 catalysts were prepared according to the protocol described above under heading A. A marginal increase in the conversion of ethanol was observed (FIG. 4). However, ethylene selectivity was observed to increase sharply, and subsequently diethyl ether formation was observed to be suppressed. Without being bound by theory, it is believed that this may suggest that ethylene is being formed via diethyl ether, and that having maximum concentrations of DEE may be a factor in forming ethylene with high yields.

### Example 4

Dehydration of ethanol in aqueous ethanol solutions over (20%) H₃PO₄ impregnated H-ZSM-5 catalyst was conducted under the same reaction conditions as those described in Comparative Example 1. H₃PO₄ modified H-ZSM-5 catalysts were prepared according to the protocol described above under heading A. The results are presented in Table 4. Except for the-ethanol concentration of 10 vol %, the dilution of ethanol was observed to not affect the catalytic performance of the catalytic system.

TABLE 4
Results of ethanol dehydration activity of aqueous ethanol
solutions on (20%) H₃PO₄ impregnated H-ZSM-5 catalyst^{a}

| Vol % of ethanol | % Conversion | % Selectivity |
|---|---|---|
| in water | of ethanol | of ethylene |
| 10 | 99.45 | 99.2 |
| 25 | 99.79 | 99.45 |
| 50 | 99.85 | 99.07 |
| 100 | 99.73 | 99.28 |

^{a}Reaction Conditions: 0.5 g catalyst, 100% ethanol, 1 atm pressure.

Under the same reaction conditions as those described in Comparative Example 1, in comparison with various H₃PO₄ impregnated ZSM-5 catalysts, it can be observed that the reaction rate is associated with the property of the catalyst material. The comparative results for this reaction over H₃PO₄ impregnated ZSM-5 and a conventional ZSM-5 catalyst are shown in FIGS. 1, 2, 3 and 4, whereby the highest conversion of ethanol (above 99%) was achieved on a H₃PO₄ impregnated ZSM-5 catalyst under the same reaction conditions as Comparative Example 1.

### Example 5

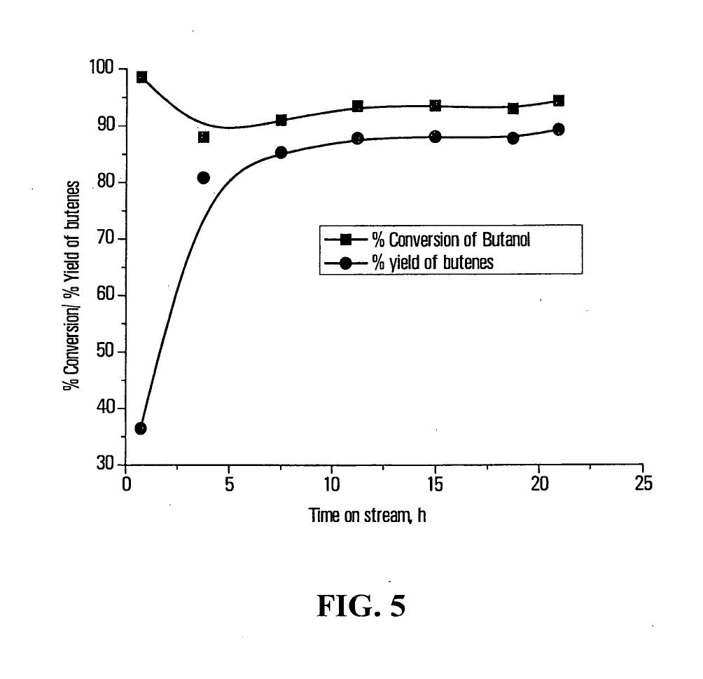

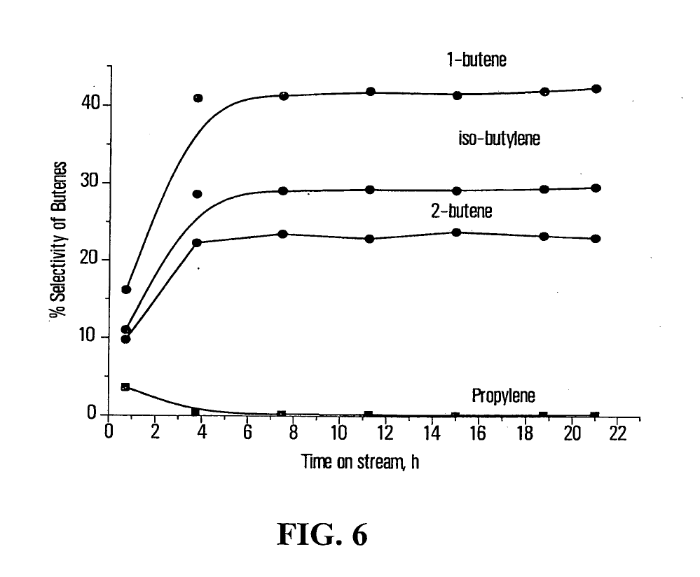

A butanol dehydration reaction over H₃PO₄ (20 wt %) modified H-ZSM-5 catalyst was conducted under the same reaction conditions as those described in Comparative Example 1 at 325° C. and 1 atm of pressure. H₃PO₄ modified H-ZSM-5 catalyst was prepared according to the protocol described above under heading A. The results are shown in FIGS. 5 and 6. A conversion of above 90% and a yield of butenes above 85% at 325° C. were observed. As can be seen from FIG. 5, no deactivation was observed after running for 21 hours.

### Example 6

Dehydration of methanol and ethanol mixtures over

H₃PO₄ (20 wt %) modified H-ZSM-5 catalyst was conducted under the same reaction conditions as those described in Comparative Example 1 at 400° C. H₃PO₄ modified H-ZSM-5 catalyst was prepared according to the protocol described above under heading A. Stable conversions of 47% and 75% for methanol and ethanol, respectively, were observed. An ethylene selectivity of about 65% was observed. The formation of propylene, butylenes and aromatics was also observed.

### CONTROL EXAMPLE

Under similar conditions as to those described in Comparative Example 1, a blank reactor without catalyst was screened at 400° C. and no ethanol conversion was observed during this study.

The vapour phase catalytic dehydration of ethanol on H-ZSM-5 catalysts was shown in FIG. 1. The reaction was carried out at 400° C. and 1 atm of pressure using 500 mg of the catalyst. Ethanol conversion was observed to reach 99% and to steadily decrease over the time. The selectivity towards ethylene was observed to also increase initially and decrease along similar lines with the conversion of ethanol. However, the selectivity towards diethyl ether was observed to increase steadily at the expense of ethylene formation. Other products, such as lower olefins (propylene and butylenes) and aromatics (benzene, toluene and xylenes), were observed to also form during the reaction. These results suggest that there is a need to develop a solid catalyst which is durable or exhibits stability over time.

Catalytic dehydration of ethanol over 20 wt % H₃PO₄ impregnated H-ZSM-5 catalyst was shown in FIG. 3. The conversion of ethanol was observed to increase with time and reach a conversion of 100%. Ethylene selectivity was observed to also follow a similar trend and achieve a yield of 98%. The H₃PO₄ impregnated H-ZSM-5 catalyst was observed to show no sign of deactivation and remained stable for 150 hours. The H₃PO₄ impregnated H-ZSM-5 catalyst has been observed to show no significant deactivation for over 250 hours. No significant amounts of formation of other products were observed.

The catalytic dehydration activity with respect to weight hourly space velocity (WHSV, h⁻¹) at 300° C. was shown in FIG. 4. As can be seen from FIG. 4, lower space velocities were observed to favour the formation of diethyl ether compared to ethylene, and at higher WHSV, the formation of ethylene was observed to be more predominant.

The process of the present invention is related to the development of solid dehydration catalysts which carry out ethanol dehydration in aqueous ethanol solutions. In this regard, the effect of ethanol concentration in water from 25 to 100 vol % was studied, and the results are shown in Table 4. No significant differences in the catalytic activity were observed by diluting the ethanol solutions in this range. It is believed that this result is significant in the sense that the catalyst composition of the invention may tolerate excess amounts of water without losing any activity.

The process of the present invention provides for the production of ethylene selectively from ethanol at appropriate reaction temperatures. The temperature during the reaction may be controlled, for example, and without limitation, between 250 to 450° C., and including any specific value within this range, such as, for example, 400° C. At low reaction temperatures, below 350° C., the formation of diethyl ether was observed to be more predominant and at higher temperatures, was observed to result in the formation of ethylene more selectively.

The present invention is particularly advantageous because the process for the selective synthesis of ethylene from aqueous ethanol solutions can be achieved on a modified ZSM-5 catalyst. As shown in Table 4, the conversion and/or selectivities were observed to not vary significantly by the dilution of ethanol in water at 350 and 400° C. (Table 4). It is believed that these results suggest that the H₃PO₄ modified H-ZSM-5 catalyst is a water tolerant solid catalyst.

It is believed that the studies suggest that the process for selective production of ethylene can be improved by adding modifying agents, including phosphoric acid, to H-ZSM-5 catalyst.

It is to be understood that any variations falling within the scope of the claimed invention and thus, the selection of a specific device or apparatus, and specific H₃PO₄ impregnated H-ZSM-5 catalysts can be determined without departing from the spirit of the invention herein disclosed.

The present invention includes isomers such as geometrical isomers, optical isomers based on asymmetric carbon, stereoisomers and tautomers and is not limited by the description of the formula illustrated for the sake of convenience.

Although the foregoing invention has been described in some detail by way of illustration and example, and with regard to one or more embodiments, for the purposes of clarity of understanding, it is readily apparent to those of ordinary skill in the art in light of the teachings of this invention that certain changes, variations and modifications may be made thereto without departing from the spirit or scope of the invention as described in the appended claims.

It must be noted that as used in the specification and the appended claims, the singular forms of “a”, “an” and “the” include plural reference unless the context clearly indicates otherwise.

Unless defined otherwise all technical and scientific terms used herein have the same meaning as commonly understood to one of ordinary skill in the art to which this invention belongs.

All publications, patents and patent applications cited in this specification are incorporated herein by reference as if each individual publication, patent or patent application were specifically and individually indicated to be incorporated by reference. The citation of any publication, patent or patent application in this specification is not an admission that the publication, patent or patent application is prior art.

1. A catalyst composition comprising a catalyst and a modifying agent, wherein the modifying agent is phosphoric acid, sulphuric acid or tungsten trioxide, or a derivative thereof, and
the phosphoric acid or derivative thereof, when present, is in an amount from greater than 10 wt % and up to 50 wt %, and the tungsten trioxide, when present, is in an amount from greater than 5 wt % and up to 50 wt %.

2. The catalyst composition according to claim 1, wherein the modifying agent is anchored to a surface of the catalyst.

3. The catalyst composition according to claim 1, wherein the modifying agent is impregnated within pores of the catalyst.

4. The catalyst composition according to claim 1, wherein the modifying agent is phosphoric acid or a derivative thereof.

5. The catalyst composition according to claim 4, wherein the phosphoric acid or derivative thereof is present in an amount of up to about 20.0 wt %.

6. The catalyst composition according to claim 1, wherein the catalyst is a bulk oxide or a zeolite.

7. The catalyst composition according to claim 1, wherein the catalyst is a bulk oxide which is alumina, zirconia, titania, silica or niobia; or a combination thereof.

8. The catalyst composition according to claim 1, wherein the catalyst is a zeolite.

9. The catalyst composition according to claim 8, wherein the zeolite is a pentasil-type zeolite.

10. The catalyst composition according to claim 8, wherein the zeolite is H-ZSM-5.

11. The catalyst composition according to claim 1, which has a Si/Al ratio of from about 20 to about 280.

12. The catalyst composition according to claim 1, which has a surface area of from about 70 to about 200 m²/g and a pore volume of from about 0.10 to about 0.17 cc/g.

13. A catalyst composition comprising a catalyst and a modifying agent,
wherein the modifying agent is phosphoric acid, sulphuric acid or tungsten trioxide, or a derivative thereof; and
wherein the catalyst composition is for selective conversion of ethanol to ethylene.

14. A catalyst composition comprising a catalyst and a modifying agent,
wherein the modifying agent is phosphoric acid, sulphuric acid or tungsten trioxide, or a derivative thereof; and
wherein the catalyst composition is for selective conversion of butanol to butylenes.

15. A process for preparing an alkene by dehydration comprising mixing one or more alcohols and optionally water and the catalyst composition as defined in claim 1.

16. The process according to claim 15, wherein the alcohol is ethanol, n-butanol or a combination of methanol and ethanol, and the dehydration reaction is conducted at a temperature of from about 250 to about 450° C.

17. A process for preparing ethylene by dehydration comprising mixing ethanol and optionally water, and the catalyst composition as defined in claim 13, and wherein the dehydration reaction is conducted at a temperature of from about 250 to about 450° C.
